(19) United States
(12) Patent Application Publication
(10) Pub. No.: US 2012/0004738 A1
(43) Pub. Date: Jan. 5, 2012

(54) Amputation Stump Warmer

(76) Inventor: Marinus Todd Westrate, Kalamazoo, MI (US)

(21) Appl. No.: 12/977,719

(22) Filed: Dec. 23, 2010

Related U.S. Application Data
(60) Provisional application No. 61/290,233, filed on Dec. 27, 2009.

(51) Int. Cl. A61F 2/78; A61F 7/00

(52) U.S. Cl. 623/36; 607/108

(57) ABSTRACT

An amputation stump warmer is disclosed herein comprising a sleeve with an inner layer and an outer layer. Amputation stump warmer further comprises an electrically conductive wire and a heating system with a rechargeable battery. The sleeve encloses an amputation stump and may be worn underneath a prosthetic limb or alone.

### RELATED U.S. APPLICATION DATA

This application is a non-provisional of and claims the benefit of the filing date of U.S. Provisional application No. 61290186 filed on 26 Dec. 2009.

### BACKGROUND

The present invention relates to prosthetics. Adjusting to life without a limb after amputation continues to be difficult for amputees. In particular, the amputee suffers from poor circulation and nerve damage in the amputation stump which causes a lower body temperature in the stump as well as great discomfort. External heaters can be used to warm the stump and increase circulation as well as provide some relief to the amputee. Several devices are known that function to portably heat a part of the body, usually via an article of clothing, such as a sock or a glove. Among these are articles of clothing equipped with various types of heating units. One such article of clothing comprises a glove with a heated insert. Another is a coat with a heating element coupled to a heating system. Advantageously, all of these devices, especially when supplied with power, would permit the user to warm a part of his or her body for an extended period of time. However, these devices are not fully effective when the part of the body is an amputation stump.

### BRIEF SUMMARY OF THE INVENTION

The known devices are not fully effective because they are not designed to work with an amputation stump and/or a prosthesis. At the heart of the present invention is the discovery that improving circulation by heating an amputation stump does not require cumbersome, ill-fitting devices that prevent free movement.

In accordance with the invention, heating an amputation stump is enhanced by utilizing a specially designed sleeve to fit over an amputation stump, both when the amputee is wearing a prosthetic limb and when he or she is not. In this manner, the present invention serves the purpose of heating the amputation stump to increase circulation while at the same time functioning as a liner between the stump and the prosthetic, if the need arises. The present invention is designed to work with any size or type of prosthetic and can be used by individuals with amputations above and below the knee, as well as above and below the elbow.

### BRIEF DESCRIPTION OF THE DRAWINGS

FIG. 1 illustrates an amputation stump warmer embodying the principles of the present invention.

FIG. 2 illustrates a cross section view of the inside of amputation stump warmer embodying the principles of the present invention.

FIG. 3 consists of FIGS. 3A, 3B and 3C, and illustrates an in-use condition of an embodiment of the present invention according to FIG. 1.

FIG. 4 illustrates a method of using an artificial limb protection system embodying the principles of the present invention.

### DETAILED DESCRIPTION

Referring now to the drawings in detail, this invention will be described by way of example and with reference to preferred embodiments thereof, however, it is to be understood that modifications and improvements may be made without departing from the scope or spirit of the invention. It should be noted that various changes and modifications to the presently preferred embodiments described herein will be apparent to those skilled in the art. Such changes and modifications may be made without diminishing its attendant advantages. It is therefore intended that such changes and modifications be included within the present invention. It should be understood that the current invention embodied herein is customizable and designed to work with the amputation stump of any limb or body part. For purposes of this specification, the term “amputation stump” is used interchangeably and refers to any part of the body that is left after a limb or projecting body part is removed, regardless of its location, shape or size.

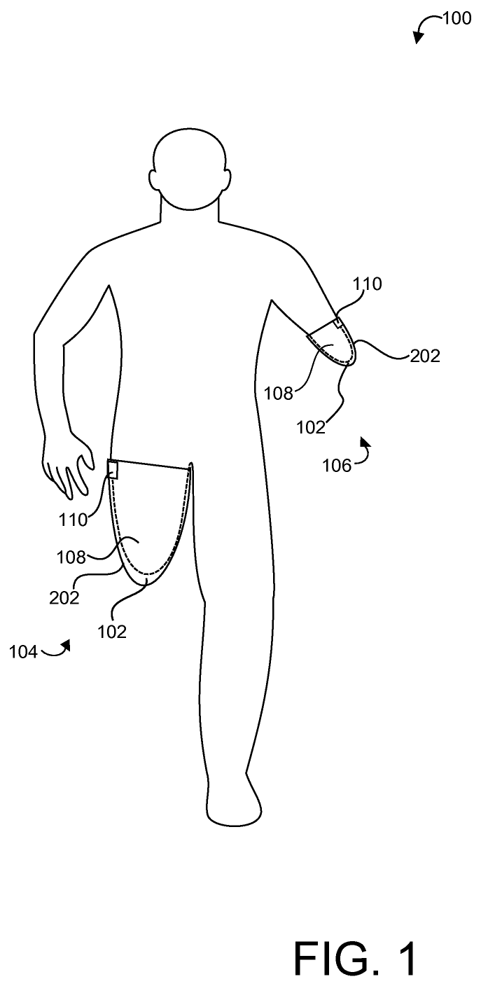

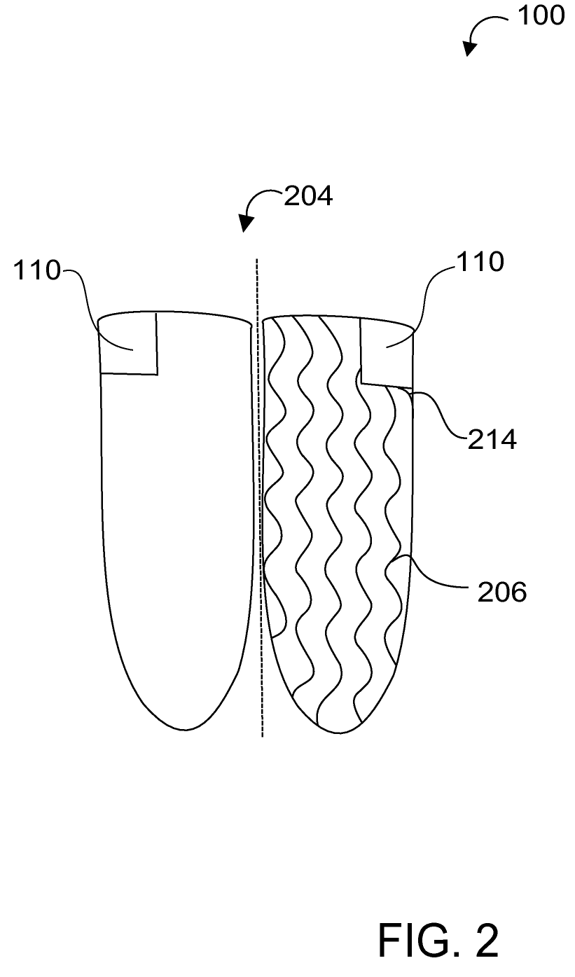

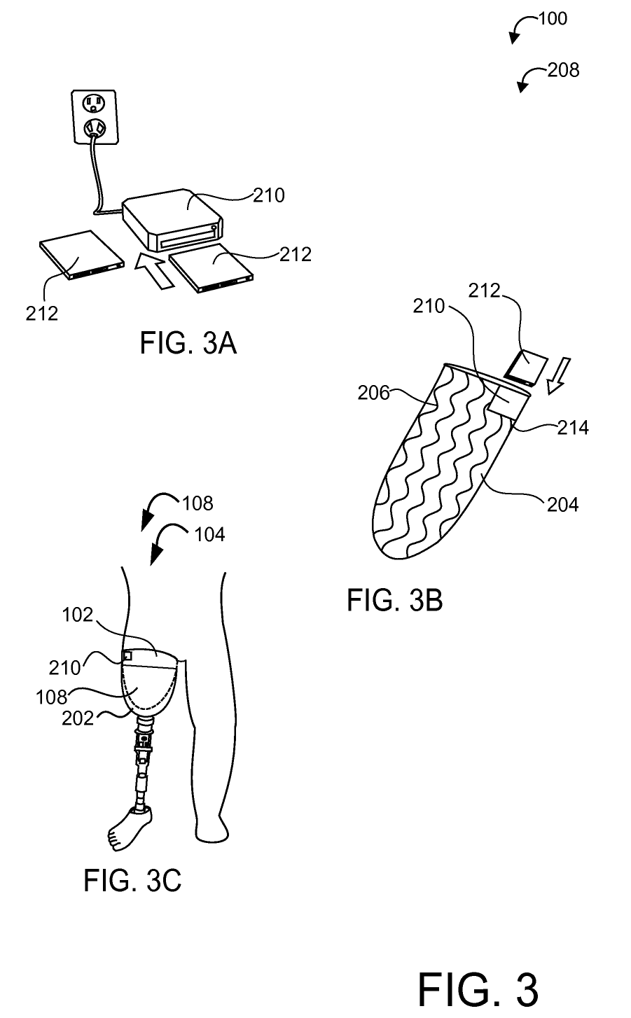

Referring now to FIG. 1, illustrating an amputation stump warmer 100 embodying the principles of the present invention. Amputation stump warmer 100 may comprise at least one sleeve 102; at least one pocket 110; and at least one heating system 208 (shown and discussed in FIGS. 2 and 3). Sleeve 102 may be a substantially cylindrical fitted cover for amputation stump 108. Sleeve 102 may further comprise outer layer 202 and inner layer 204 (shown and discussed in FIG. 2). When in use, inner layer 204 may be in direct contact with the skin of amputation stump 108, while outer layer 202 may be in direct contact with the inner shell of a prosthetic. In alternative embodiments, outer layer 202 may be exposed. In this manner, amputation stump warmer 100 may be portable such that the user is capable of free movement while wearing amputation stump warmer 100 in conjunction with his or her prosthesis.

Sleeve 102 may further comprise at least one pocket 110. Pocket 110 comprises a substantially pouch-like configuration for removably receiving at least one battery 212 (shown and discussed in further detail in FIG. 3). In a preferred embodiment, pocket 110 may be located on the portion of outer layer 102 furthest from the user's body as shown. In this manner, pocket 110 prevents battery 112 from chafing and discomfort when sleeve 102 is worn. Preferably, sleeve 102 may be of a sufficient size to extend over the length amputation stump 108 and up the remaining limb such that a portion of it remains exposed should the user wish to use amputation stump warmer 100 with his or her prosthesis. In this manner, pocket 110 may be located substantially above the topmost portion of the user's prosthesis (Shown in FIG. 3).

Finally, sleeve 102 may be made of any stretchable/elastic fabric suitable to provide a smooth, snug fit against amputation stump 108. In this manner, sleeve 102 is capable of stretching up over a substantial portion of amputation stump 108 without slouching, wrinkling, bunching, and /or falling down as it is worn by itself or under a prosthetic. Additionally, sleeve 102 preferably comprises a stretchable fabric also suitable for moisture wicking and/or heat diffusion. Examples of such fabrics include, but are not limited to, such as cotton, wool, natural or synthetic fibers, fabric blends, or other suitable material. In this manner, when the user wears sleeve 102.

By way of example only, FIG. 1 depicts amputation stump warmer 100 configured to cover amputation sump 108 above the knee 104 and above the elbow 106. In alternative embodiments, amputation stump warmer 100 may be configured to fit amputation stump 108 below the knee or below the elbow, such as, for example, and ankle or a wrist (not shown). Further, the dimensions of sleeve 102 may be configured to work with any size or shape of amputation stump. In this manner, the user is not limited to a particular type of prosthetic.

Referring now to FIG. 2, illustrating a cross section view of the inside of sleeve 102 embodying the principles of the present invention. As mentioned above, amputation stump warmer 100 may comprise at least one heating system 208. Heating system 208 may comprise at least one heating element 206; at least one case 210 (shown in FIG. 3); and at least one battery 212 (shown in FIG. 3). In this manner, amputation stump warmer 100 may function to warm amputation stump 108, improve circulation, reduce discomfort, function with or without the user's prosthetic, and permit the user to move freely about while wearing it.

The heating of amputation stump warmer 100 may be achieved using heating system 208. Case 210 may comprise any type of housing known in the art sufficient to function as a receptacle and/or charging means for battery 212. Battery 212 may comprise any suitable type of rechargeable and/or non rechargeable heating system sufficient to function with a portable heat source, such as, for example, rechargeable nickel metal hydride batteries, alkaline or dry cell batteries, and the like. In a preferred embodiment, heating system 208 comprises at least one rechargeable lithium ion (“Li-ion”) battery.

Heating system 208 may further comprise heating element 206. Heating element 206 may comprise a plurality of electrically conductive wires known in the art, such as those described in U.S. Pat. No. 6,713,724. Heating element 206 may run the length or width of sleeve 102. Further, heating element may be positioned between inner layer 204 and outer layer 202 as shown. In this manner, sleeve 102 may function to house heating element 206 and protect the user's skin from direct exposure of heating element 206. Heating element 206 may further be an interconnected single length of conductive material configured in a non-linear pattern, such as a wave as shown. In an alternative embodiment, heating element 206 may comprise a plurality of interconnected wires configured in a non-linear pattern.

Finally, heating element 206 may comprise terminal end 214 which may be connected to battery 212. In a preferred embodiment, terminal end 214 may be substantially located near the top of sleeve 102 and the bottom portion of pocket 110 as shown. In this manner, when battery 212 is connected to heating element 206, electric energy is converted to thermal energy and travels the length of heating element 206, which radiates from heating element 206 through sleeve 102 to the skin of amputation stump 108, thus functioning to heat amputation stump 108. Finally, amputation stump warmer 100 may be turned on and off by connecting and/or disconnecting battery 212 to/from heating element 206. When battery 212 dies, the user simply replaces it with a fully charged one.

Referring now to FIG. 3 consisting of FIGS. 3A, 3B, and 3C, illustrating an in-use condition of an embodiment of the present invention according to FIG. 1. FIG. 3A illustrates two (2) battery 212; and one case 210. When the user wishes to use amputation stump warmer 100, he or she inserts battery 212 into case 210, which may be connected to an electrical outlet as shown, and charges battery 212. In alternative embodiments (not shown) the user may use a disposable, non-rechargeable battery. As illustrated by FIG. 3B, the user may then insert battery 212 into pocket 210 and connect to terminal end 214, thus powering heating system 208 to create heat.

By way of example, FIG. 3C illustrates amputation stump warmer 100 as worn on amputation stump 108 above the knee 104 with a prosthetic leg 220. The user may stretch sleeve 102 over amputation stump 108 and subsequently attach prosthetic 220. Sleeve 102 and pocket 210 containing battery 212 rests against the remaining portion of the user's limb above the top most portion of prosthetic 220 as shown. As mentioned previously, it is contemplated within the current invention that amputation stump warmer 100 may be worn over any size or type of amputation stump, such as, for example, an ankle or shoulder without departing from the scope and spirit of the invention.

Finally, amputation stump warmer 100 may comprise a kit 400 including sleeve 102; at least one heating system 208; and a set of user instructions (not shown). Amputation stump warmer 100 may be manufactured and provided for sale in a wide variety of sizes and shapes for a wide assortment of applications including refrigerated versions and non-refrigerated versions. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, parts may be sold separately, etc., may be sufficient.

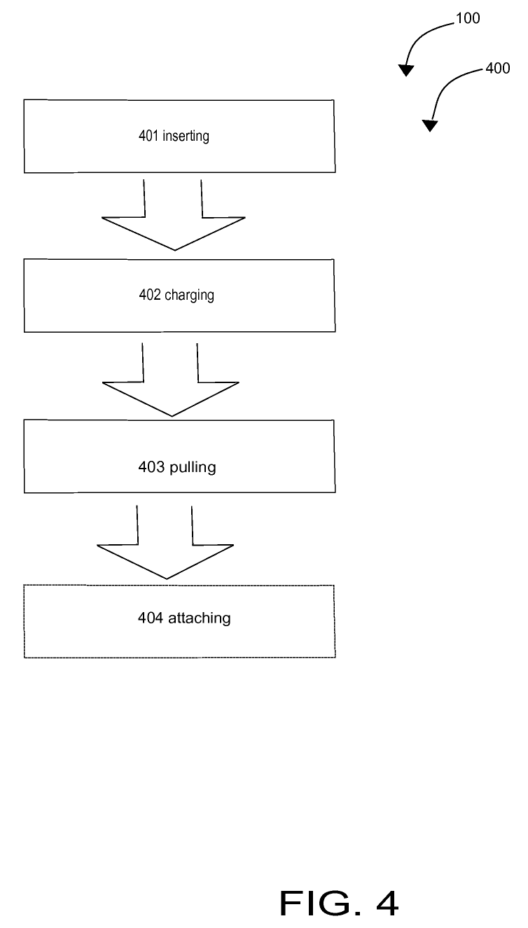

FIG. 4 is a flowchart illustrating a method of use 400 for amputation stump warmer 100 embodying the principles of the present invention. Method of use 400 for operating amputation stump warmer 100 preferably comprises the steps of: step one 401 inserting battery 212 into case 210; step two 402 charging battery 212; and step three 403 pulling sleeve 102 over amputation stump 108. Method of use 400 may further optionally comprise step four 404 comprising attaching a prosthetic limb. It should be noted that step 404 is an optional step and may not be implemented in all cases. Optional step 404 of method 400 is illustrated using dotted lines in FIG. 4 so as to distinguish it from the other steps of method 400.

It should be noted that the steps described in the method of use can be carried out in many different orders according to user preference. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

From the foregoing description, it should be appreciated that a preferred embodiment and a method for warming an amputation stump is provided and present significant benefits that would be apparent to one skilled in the art. Furthermore, it should be appreciated that a vast number of variations in the embodiments exist. The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

What is claimed is new and desired to be protected by Letters Patent is set forth in the appended claims:

1. An amputation stump heating appliance comprising:
at least one sleeve having an inner layer and an outer layer;
at least one electrically conductive wire;
at least one heating system having a battery;
wherein said at least one sleeve encloses an amputation stump; and
wherein said heating system said battery connects to said electrically conductive wire.

2. The amputation stump heating appliance of claim 1 wherein said at least one sleeve comprises a pocket.

3. The amputation stump heating appliance of claim 2 wherein said battery is removably received by said pocket.

4. The amputation stump heating appliance of claim 1 wherein said at least one sleeve comprises a flexible cover.

5. The amputation stump heating appliance of claim 4 wherein said flexible cover comprises stretchable fabric.

6. The amputation stump heating appliance of claim 4 wherein said flexible cover shrouds an amputation stump.

7. The amputation stump heating appliance of claim 1 wherein said at least one electrically conductive wire is positioned between said inner layer and outer layer.

8. The amputation stump heating appliance of claim 1 wherein said battery is rechargeable.

9. The amputation stump heating appliance of claim 1 wherein said heating system further comprises a charger.

10. The amputation stump heating appliance of claim 9 wherein said charger provides energy to said battery.

11. The amputation stump heating appliance of claim 1 wherein said at least one conductive wire is connected to said battery.

12. The amputation stump heating appliance of claim 11 wherein said battery and said at least one electrically conductive wire when connected create thermal energy, said thermal energy radiating through said at least one sleeve to warm said amputation stump.

13. An amputation stump heating system comprising:
an amputation stump heating appliance,
having at least one sleeve, said at least one sleeve having an inner layer, an outer layer, and a pocket, said at least one sleeve and enclosing at least one amputation stump;
said pocket removably receives a battery;
at least one electrically conductive wire positioned between said inner and outer layers; and
a heating system within or in communication with said amputation stump heating appliance to warm an amputation stump;
wherein said charger serves to provide power to said at least one battery
wherein said battery is removably received by said pocket and wherein said battery is connected to said at least one electrically conductive wire.

14. The amputation stump warming appliance of claim 13 further comprises a kit including said amputation stump warming appliance; and a set of user instructions.

15. A method of operating an amputation stump heating appliance comprising the steps of:
charging a battery;
putting on said sleeve;
inserting battery into said pocket; and
connecting said battery to said electrically conductive wire.

16. The method of operating an amputation stump heating appliance of claim 15 further comprising the step of attaching a prosthetic limb over said sleeve.

17. The method of operating an amputation stump heating appliance of claim 15 further comprising the stem of removing said battery.
pulling on a sleeve having at least one pocket and said sleeve enclosing at least one electrically conductive wire;
inserting said battery into said at least one pocket; and
connecting said at least one battery to said electrically conductive wire.

18. The method of operating an amputation stump heating applications of claim 14 further comprising the step of attaching a prosthesis.
